(19) United States
(12) Patent Application Publication
(10) Pub. No.: US 2021/0172936 A1
(43) Pub. Date: Jun. 10, 2021

(54) Methods for Culturing and for Detecting Stealth Adapted Viruses

(71) Applicant: William John Martin, South Pasadena, CA (US)

(72) Inventor: William John Martin, South Pasadena, CA (US)

(21) Appl. No.: 16/707,961

(22) Filed: Dec. 9, 2019

(51) Int. Cl. G01N 33/50; C12N 5/078; C12N 7/00; G01N 21/64

(52) U.S. Cl. CPC G01N 33/5094; C12N 5/0634; G01N 2021/6439; G01N 21/6428; C12N 2500/90; C12N 7/00

(57) ABSTRACT

Stealth adapted viruses differ from the conventional viruses from which they are derived in not evoking an inflammatory response. This can occur because of the deletion or mutation of the genes coding for the relatively few virus components, which are normally targeted by the cellular immune system. As part of the stealth adaptation process, exchanges can occur between some and possibly all of the sequences of the initiating virus and sequences of both cellular and bacteria origin. A description is provided on the culturing of stealth adapted viruses. A characteristic feature of cultured stealth adapted virus infected cells is the accumulation of intracellular materials, which will fluoresce under ultraviolet (UV) light in the presence of certain dyes including neutral red and acridine orange.

### RELATED PUBLICATION BY THE APPLICANT

1. Martin W J, Zeng L C, Ahmed K, Roy M (1994). Cytomegalovirus-related sequences in an atypical cytopathic virus repeatedly isolated from a patient with the chronic fatigue syndrome. American Journal of Pathology 145: 441-452.

2. Martin W J, Ahmed K N, Zeng L C, Olsen J-C, Seward J G, Seehrai J S (19950. African green monkey origin of the atypical cytopathic ‘stealth virus’ isolated from a patient with chronic fatigue syndrome. Clinical. Diagnostic Virolology 4: 93-103.

3. Martin, W.J. (2003) Stealth Virus Culture Pigments, A Potential Source of Cellular Energy. Experimental Molecular Pathology, 74, 210-223.

4. Martin W J (2014). Stealth Adapted Viruses; Alternative Cellular Energy (ACE) & KELEA Activated Water. Author House, I N. pp 321. ISBN 978-1-4969-0496-6.

5. Martin W J and Laurent D (2015) Homeopathy as A Misnomer for Activation of the Alternative Cellular Energy Pathway, Evidence for the Therapeutic Benefits of Enercel in a Diverse Range of Clinical Illnesses. International J Complementary & Alternative Medicine 2(1), 00045

6. Enhancing the Alternative Cellular Energy (ACE) Pathway with KELEA Activated Water as Therapy for Infectious Diseases (Submitted)

7. Martin W J (2019) Renegade Cellular and/or Bacterial Genetic Sequences in Stealth Adapted Viruses. International journal complimentary and alternative Medicine. (Submitted)

### RELATED PATENTS AND PATENT APPLICATIONS BY THE APPLICANT

Stealth virus detection in the chronic fatigue syndrome. U.S. Pat. No. 5,756,281 Issued May 26, 1998.

Stealth virus detection in the chronic fatigue syndrome. U.S. Pat. No. 5,985,546 Issued Nov. 16, 1999.

Method of Enhancing the Alternative Cellular Energy Pathway in Humans and Animals Using Wearable Items That Contain KELEA Activated Water. Submitted Feb. 18, 2019. application Ser. No. 16/278,712

Method of Detection and Measurement of a Life Force Energy, Also Known as KELEA in Liquids & Other Materials. Submitted May 23, 2019. application Ser. No. 16/421,344

Using paper with heightened KELEA to Enhance the alternative cellular energy pathway. Submitted Jul. 10, 2019. application Ser. No. 16/508255

### STATEMENT REGARDING FEDERALLY SPONSORED RESEARCH OR DEVELOPMENT

Not applicable: No Federal funding was received in support of this patent application.

### FIELD OF THE INVENTION

The invention is within the fields of virology and chronic illnesses, especially those chronic illnesses in which there are neurological and psychiatric symptoms. The invention specifically addresses the utility of a specialized cell culturing technique for the detection of a grouping of viruses, which typically do not evoke inflammation because of an immune evasion mechanism referred to as stealth adaptation. These viruses may also acquire some of their genetic information by incorporating what are being referred to as “renegade” cellular and/or bacterial sequences. The renegade sequences can substitute for some and possibly all of the genetic sequences in the originating virus, which may, therefore, be no longer detectable using routine serological and molecular markers that are diagnostic of the particular originating virus. Infected cells do, however, produce atypical materials, which will fluoresce when exposed to certain dyes. This approach, in conjunction with the specialized virus culturing techniques, allows for the detection of stealth adapted viruses in patients with a wide range of illnesses, including many neurological and psychiatric illnesses.

### BRIEF SUMMARY OF THE INVENTION

Apart from the Applicant, researchers have failed to obtain evidence of virus infections in patients with many types of chronic neurological and psychiatric illnesses. This failure has largely resulted from i) The belief that the absence of brain inflammation in these illnesses essentially precludes an infectious agent. ii) The use by these other investigators of inadequate methodologies to successfully culture viruses from the blood, cerebrospinal fluid, and tissues obtained from the patients. iii) An expectation that virus infected cells will inevitably display molecular and/or serological markers, which can be equated with those that characterize a known human or animal virus.

The Applicant has addressed the first issue by describing an immune evasion mechanism, which is defined as stealth adaptation. Stealth adapted viruses differ from the viruses from which they are derived in having deletions or mutations in the genes coding for the relatively few virus components normally targeted by the cellular immune system. The Applicant has also addressed the inadequate culturing techniques being used by others. He initially did so in U.S. Pat. Nos. 5,756,281 and 5,985,546), which have been previously awarded to the Applicant. These patents, which are incorporated herein by reference, describe a culture method in which the primary indication for the detection of stealth adapted viruses is the development of a cell damaging or cytopathic effect (CPE). The CPE develops in indicator cells, which are co-cultured with a combination of mononuclear and polymorphonuclear cells from the patients. The present application provides for improved culture methodology using blood and other materials collected from patients. More importantly, the present application describes a fluorescence-based method, using certain dyes, including acridine orange and neutral red, which provide significant improvements over relying upon on the appearance of a discernable CPE to determine that a stealth adapted virus culture is positive. The fluorescence-based method can also assist in the further characterization of stealth adapted viruses and in the evaluation of potential anti-virus therapies. Additional research findings have also led to a greater understanding of what constitutes stealth adapted viruses and how relying upon the detection of conventional serological and molecular markers for identifying viruses and other pathogens can be misleading. This can occur due to reformation of a replicative virus following the exchange of some of the original virus components with other genetic elements, including genetic sequences of cellular and of bacterial origins. This information is included in the recently submitted article entitled “Renegade Cellular and/or Bacterial Genetic Sequences in Stealth Adapted Viruses,” which should be shortly posted online and is included in this application by reference. The utility of a more reliable method to detect stealth adapted virus infections in association with many neurological and psychiatric illnesses, and possibly many cancers, will open major pathways to improved therapies and to disease prevention.

### BRIEF DESCRIPTION OF THE DRAWINGS

Not Applicable and none included

### DETAILED DESCRIPTION OF THE INVENTION

The earlier U.S. Pat. Nos. 756,281 and 5,985,546, were submitted on Nov. 23, 1993 and May 26, 1998, respectively. The primary observation was that the co-culturing of patients' white blood cells with human or animal indicator cells would lead to a foamy vacuolated CPE in the indicator cells, with more or less evidence of cell fusion (syncytia formation). The blood cells comprised mononuclear cells, isolated from centrifuged heparinized whole blood layered onto ficoll-hypaque density medium. These cells were combined with polymorphonuclear cells (neutrophils, eosinophils and basophils) drawn from the buffy coat, which forms as a layer over the erythrocytes (red blood cells) and below the ficoll-hypaque in the centrifuged blood. Invariably, there was significant erythrocyte contamination of the polymorphonuclear cells. After washing the patients' combined cell populations by centrifugation, the cells were added to culture tubes containing adherent indicator cells. It was necessary to remove most of the contaminating erythrocytes by repeated adding and decanting of tissue culture medium to and from the culture tubes containing the indicator cells. The indicator cells lines included human foreskin fibroblasts (MHRF), human MRC-5 cells, and rhesus monkey kidney cells (RhMK). These cells are commercially available in test tube shaped, roller culture tubes. An important observation leading to the earlier patent applications was the need to regularly replace at 24 to 72-hour intervals, the tissue culture medium in the culture tubes containing the indicator cells. This was necessary for the CPE to progress and to prevent a healing process from occurring. The CPE formation was also enhanced by 30% supplementing the media with filtered supernatants of cultures of human cytomegalovirus, referred to as virus enhancing medium (VEM). Another procedure to enhance the development of the CPE was to detach the indicator cells from the culture tubes and co-centrifuge the detached cells with the patient's cells before adding the combined pelleted cells back to the culture tube.

Several major improvements have been made in the earlier virus culturing procedure since the time of the second patent submission. First, I learned that it was preferable to use only the mononuclear cells rather than combining these cells with buffy coat polymorphonuclear cells. The polymorphonuclear cells are inherently somewhat toxic to the indicator cells. This modification also eliminated erythrocyte contamination. The next major advance was to freeze the collected mononuclear cells, to be thawed when later needed for setting up the cultures. Beyond the added convenience, secreted factors from the viable mononuclear cells were delaying the development of the CPE. The use of frozen-thawed cells leads to a more intense CPE, usually observable within a week. This change also allows for the omission of two additional steps described in the earlier patents. These steps were the benefits of adding the virus-enhancing-medium (VEM) obtained from cultures of human cytomegalovirus, and the time-consuming practice of co-centrifuging the mononuclear cells with detached indictor cells, with the need to replant the cells back into the culture tubes. Another major improvement, referred to briefly in the second patent, is the avoidance of using fetal calf serum in the culture medium. It is far preferable to culture the indicator cells plus thawed extracts of the patient's mononuclear cells in serum-free media, such as X Vivo-15, available from Lonza-BioWhittaker. This omission of fetal calf serum removes a potential confounding factor in interpreting results using both fluorescence-based and molecular-based assays. including the polymerase chain reaction (PCR).

The PCR assay was decisive in establishing the origin of the prototype stealth adapted virus cultured from a patient with the chronic fatigue syndrome (CFS). The PCR assay clearly showed that the virus was derived from African green monkey simian cytomegalovirus (SCMV). A virus cultured from the CSF of a patient with a bipolar psychosis was also shown by PCR to be derived from SCMV, as have some other isolates. The SCMV origin of some stealth adapted viruses is explainable since live polio vaccines were produced in the kidney cells of African green monkeys. At an earlier time, most of the African green monkeys were infected with SCMV. DNA of SCMV is detectable in some of these previously licensed polio vaccines.

The prototype stealth virus cultured from the CFS patient was further molecularly characterized using both PCR and DNA cloning. In addition to SCMV-related genetic sequences, the virus has previously incorporated several genetic sequences from the human cellular genome and from bacteria. The cellular-derived sequences are primarily within the non-coding regions of the human genome. It is likely that non-coding long RNA transcripts from the corresponding regions in the monkey genome had earlier hybridized with fragments of the virus. These RNA sequences were then incorporated into the virus replication. As the virus passed into humans, there was a likely further exchange of the monkey sequences with human cellular sequences. Over time, the cellular sequences can come to displace some if not most of the sequences in the initiating virus. This sequence exchange can also occur with bacterial sequences, either in a eukaryotic cell or possibly in an infected bacterium. The bacteria-derived sequences include open reading frames with the presumed production of proteins. Both the cell-derived and the bacteria-derived genetic sequences have undergone further mutations, including recombination. The working hypothesis is that the bacteria proteins are providing components to help in creating a virus capsid. They are also likely contributing to a partial defense against the virus CPE by acting as alternative cellular energy (ACE) pigments.

Different cultures from CFS patients and from patients with a range of neurological and psychiatric illnesses respond differently when screened using the PCR assay. While some cultures are still positive using similar PCR assays in the sense that PCR products are generated, the only sequences being amplified are altered cellular sequences. In one culture, on human cells, the amplified cellular sequence had previously originated from a rhesus monkey genome. Sequences of conventional viruses have yet to be detected in some of these cultures. This is consistent with the variable loss of genetic sequences from the initiating viruses in the formation and evolution of stealth viruses. This helps underscore the importance of a reliable screening assay to detect the presence of stealth viruses. It is no longer sufficient to rely on the detection of the serological or molecular markers of known human or animal viruses.

An important observation in the culturing of stealth adapted viruses is that once CPE appears, it is not invariably progressive. With the single exception of cultures of the rarely encountered molluscum contagiosum virus, all of the conventional viruses cultured in clinical diagnostic virology laboratories will show the continued progression of CPE over time. In other words, once the CPE caused by conventional (non-stealth adapted) viruses begin, the cellular damage becomes progressive and, increasingly apparent. But for stealth adapted virus cultures, especially, if the cultures are not refed, the early appearance of CPE can remain somewhat stationary over several days and it will then typically regress. Fortunately, some of the previously abnormal appearing cells will have accumulated intracellular materials, which can be detected using the fluorescence-based assays described in this application.

The presence of these materials can be explained as follows: A feature of virus infection in all cells is the diversion from the production of normal cellular proteins to the overproduction of viral coded proteins. These proteins can trigger an unfolded protein response (UPR) that can potentially impede the production of more proteins by the cell. Certain of the gene products of herpes simplex virus, human cytomegalovirus and probably other viruses downregulate the UPR. The viral proteins continue to be increasingly synthesized leading to increasing cell damage. A more protracted response generally occurs in cells infected with stealth adapted viruses. Proteins and other virus components can accumulate to higher levels without the cell degenerating. Moreover, it is likely that some of the extraneous cellular and bacterial sequences incorporated into the stealth adapted virus genome are adding to the accumulating proteins. Altogether, these accumulated proteins can interact with dyes, such as neutral red and acridine orange, with the combination being detectable on the basis of ultraviolet (UV) light fluorescence.

Neutral red dye is a supravital stain, meaning that it can stain living cells. This dye does not stain dead cells. It had previously been used in virology laboratories mainly as a convenient way to identify plaques or areas of dead cells cultured in Petri dishes in distinction to the adjacent and surrounding red-staining living cells. There was a single report noting that early during the culturing of human cytomegalovirus, that the infected cells visibly stained more intensely red than the adjoining and surrounding non-infected cells. A second occasional use of neutral red dye in the early days of virology was to illuminate the neutral red treated virus cultures with either white light or more specifically green light at the absorption maxima of neutral red dye. The neutral red dye would emit light at a lower wavelength, but in doing so would lead to the production of free radicals. The free radicals would then react with the virus nucleic acids, leading to loss of the virus infectivity. This process is referred to as photodynamic therapy. It was extended to efforts to suppress herpes simplex virus infections in patients using neutral red dye with white light illumination. While an initial report was positive, subsequent reports failed to show any benefits of this form of therapy. A separate observation by Dr. Jon Stoneburner was that if a UV light was used instead of a white light, then the herpes lesions would indeed heal more quickly. An incidental finding of Dr. Stoneburner and confirmed by the Applicant ,was that the herpes skin lesions treated with neutral red dye and illuminated with UV light would brightly fluoresce a yellowish color. This was attributed by Dr. Stoneburner to a direct interaction of the dye with herpes virus particles. Subsequently, the Applicant showed that the UV fluorescence is due to the interaction of the dye with ACE pigments, which are locally produced in response to the herpes virus infection.

Acridine orange was been historically used in virus cultures in conjunction with UV illumination. The main reason for its use was the finding that while double-stranded DNA will fluoresce green when exposed to acridine orange, single-stranded RNA will fluoresce red. It was, therefore, particularly useful to detect RNA viruses, as increased red fluorescence in the cytoplasm of virus infected cells. As with neutral red dye, there has also been interest in the potential use of acridine orange in the phototherapy of various lesions. There has been less acknowledgement of acridine orange interacting with non-nucleic acid components.

With regards to this Application, the materials being formed in cells infected with the SCMV-derived stealth adapted virus from the original CFS patient virus are weakly directly fluorescent when exposed to ultraviolet (UV) light and mildly phosphorescent when exposed to white light. More dramatically, the infected cells fluoresced under ultraviolet (UV) light illumination (365 nm). The materials in the cells also greatly enhance and alter the spectrum of acridine orange fluorescence (metachromasia). The observations were confirmed using other positive stealth virus cultures. Specifically, final concentrations of approximately 0.01% both neutral red and acridine orange dyes will evoke easily discernable fluorescence in stealth virus infected cells. Especially with acridine orange, the infected cells can show a dramatic combination of intracellular bright green, yellow, and red particles, many of which can also be seen extracellularly.

As noted earlier, the only sequences identified using the PCR assays on several positive cultures from CFS patients are of cellular origin. This finding is consistent with the evolving concept of replacements of most and even possibly all of the original virus sequences with stretches of cellular and/or bacterial genetic sequences. The term “renegade viruses” is going to be used to aptly describe such viruses. The replacement sequences become part of the replication process, possibly mediated by endogenous reverse transcriptase enzymes. The substituted sequences become part of the replicative process and can be transmitted as infectious virus, even between species. This was shown by the finding of a rhesus monkey cellular sequence in a stealth adapted virus culture from a human patient.

While the PCR assays yield highly variable results on the cultures from different patients, a consistent finding is the altered UV fluorescence using acridine orange, neutral red, and some other fluorescent stains. An advantage of the neutral red dye is that it can be used to stain living cells, therefore, can be used repeatedly. The positive fluorescence reaction is retained even with the infected cells beginning to show lessening of the CPE. It is, therefore, a useful replacement for relying upon the development of CPE as the primary measure of a positive stealth adapted virus culture.

Stealth adapted viruses with renegade cellular and/or bacterial sequences would be commonly missed using assays, which are based primarily on the detection of sequences or proteins of known human and animal viruses. Screening cultures for altered fluorescence using suitable dyes provides a more reliable and generic screening method for the detection of replicating stealth adapted and renegade viruses.

More detailed description: A suitable assay involves the collection of a biological sample, such as blood, cerebrospinal fluid, fine needle aspirate of tissues, including lymph nodes and tumors. Blood is best collected in heparin tubes, such as the 8 ml green top sodium heparin tubes provided by Becton Dickerson. In one protocol, 5 ml of the whole blood is layered onto 3 ml of ficoll-hypaque in a tube, which is centrifuged at 1,000 to 1,500 rpm for 20 to 30 minutes, depending upon the centrifuge. Mononuclear cells separate as a band above the ficoll-hypaque with the erythrocytes and polymorphonuclear cells moving to the lower region of the tube, below the ficoll-hypaque. The collected mononuclear cells are added to a tube containing sterile saline and pelleted by centrifugation. The cells are then aliquoted into two or more small tubes which are capped. The tubes are placed into a −20° freezer. Indicator cells, including human foreskin fibroblasts (MRHF) and/or the human cell line MRC-5, are commercially available in either 10 ml roller culture tubes or in small shell vials with a coverslip. They are supplied in Dulbecco's Modified Eagle's Medium (DMEM). Before use, the DMEM medium in the cell containing tubes or shell vials is replaced with a serum-free medium, such as X-vivo 15 (Lonza-Bioscience). A tube of the aliquoted frozen cells is removed from the freezer and contents are thawed and added to one or more of the tubes or shell vials with the indicator cells. The tubes are placed into a 37° incubator, preferably on a slowly rotating wheel. The shell vials are placed directly into the incubator. The medium is replaced within 24 hours and, thereafter, at least at 48-hour intervals. The cells are observed under phase contrast for cellular changes. At 7 days, either the floating/detached cells are collected and cytocentrifuged onto one or more glass slides. The cells are examined for phosphorescence following exposure to white light, direct ultraviolet (UV) fluorescence, and most definitively fluorescence upon the addition of neutral red dye, acridine orange dye, or other suitable fluorescent dye. The neutral red and acridine orange dyes are typically diluted to a concentration between 0.005 and 0.05%, depending upon the sensitivity of the fluorescent microscope. Alternatively, the staining can be done on the adherent cells on the coverslips in shell vials. Depending upon the source of the neutral red dye a low concentration of the dye will not destroy the cultured cells and the cover slips can be placed back into the shell vials. This allows for continuing studies at a later time. Note it is also somewhat preferable to use phenol red free X-vivo 15 medium, which is commercially available.

A positive assay comprises particulate intracellular fluorescence. It can be accompanied by extracellular particulate fluorescence. If sufficient fluid remains, the extracellular particles will commonly display kinetic movements. The materials being detected are ACE pigments. The production of ACE pigments is a surrogate marker for the presence of stealth adapted/renegade viruses. Other stains can be used for regular microscopic examination of the altered cells and extracellular particles. Controls include tubes to which no frozen-thawed mononuclear cells were added or frozen-thawed mononuclear cells obtained from asymptomatic, non-virally infected individuals.

The positive fluorescence of materials obtained from the stealth adapted virus infected cultures is helpful in further isolation and characterization of the ACE pigments. The characterization can include gas chromatography-mass spectroscopy (GC-MS), X-ray energy-dispersive X-ray spectroscopy, and immunological reactivity. Additionally, supernatant DNA can be cloned for sequence analysis. Understanding the materials in individual patients can be used to develop more precise assays for the actual extraneous materials produced by the patient.

A major use of the assay is to determine the effectiveness of activated fluids in suppressing the formation of ACE pigments. The term activated is used in the context of the Applicant's research in which he has defined a force that he calls KELEA, an abbreviation for Kinetic Energy Limiting Electrostatic Attraction. In this regard, the X-vivo 15 and other media used can be screened for prior KELEA activation, which can occur even from being stored in the vicinity of a KELEA activated fluid or in a KELEA enhanced environment. As discussed in the pending patent application Ser. Nos. 16/278712, 16/421344, and 16/508255, KELEA activity in fluids can be assessed by the motion of small particles of lidocaine and other compounds sprinkled onto a sample of the fluid transferred to small dishes. The examination of the particles is best done microscopically but can also be seen without magnification. As also discussed in the submitted patent applications, cellulose containing materials, including paper, can attract and remove KELEA from activated fluids. A useful addition to the stealth adapted virus assay is, therefore, to place a folded sheet of sterile, clean paper a bottled of the X-vivo 15 or other culture medium intended to be used in the stealth virus culture assay for sufficient time (usually well less than 30 minutes) to reduce its KELEA level, such that sprinkled lidocaine particles remain visibly stationary.

The other major utility of the assay is to monitor the effectiveness of in vivo therapies, including the internal and external administration of KELEA activated water. to stealth virus culture positive patients. Other means are available to boost the ACE pathway and their effectiveness can be monitored by periodic virus detection using the approach described in this application.

The above described procedure can be easily adapted to high throughout screening using automated cultivation and fluorescence detection procedures. Also, many of the improvements noted in this application are extensions on the previously approved patents in which the appearance of a foamy cytopathic effect was the endpoint. It is still advisable to examine the cells for CPE, although more information can be gained by staining the cells using hematoxylin and eosin (H&E), Giemsa, periodic acid Schiff (PAS), and Stains-All dyes.

Although the description of the culture method is for heparinized blood samples, essentially similar assays can be applied to many other clinical specimens of human and animal origin. With respect to blood samples, in severely affected individuals, stealth adapted viruses can be cultured from the plasma, polymorphonuclear cells, and even from erythrocytes, to which the circulating viruses can attach. Cerebrospinal fluids can be tested directly or after centrifugation of any cellular content. Urine, saliva and sputum can also be tested, as can biopsied tumor cells. The indications are that stealth adapted viruses are a major contributing factor to chronic illnesses, and especially of those illnesses in which there are neurological and psychiatric symptoms. The major advance with the studies is a new understanding of what constitutes a virus. The research has helped deemphasize the requirement to identify serological and molecular markers of known human and animal viruses. Rather, substituted renegade sequences of cellular and bacteria origins can substitute for the components in the originating virus. The reformed virus can replicate and can pass between cells and between individuals, even across species. The infected cells will typically produce materials, which can be detected using relatively simple fluorescence assays. The utility of this assay and of modifications based on the description of this assay, should lead to improved diagnoses, prevention and therapy of many types of human and animal illnesses.

1. A method of detecting a stealth virus comprising culturing a sample under conditions in which any stealth virus in said sample is able to induce in the cells in which the virus is cultured, the formation of intracellular particulate materials, which will fluoresce under ultraviolet light illumination when the cells are exposed to an appropriate dye, thereby, providing a means of detecting the presence of said stealth virus in the sample.

2. The method of claim 1, in which the culturing is performed by:
(a) inoculating a permissive cell line with a sample; and (b) detecting the formation of intracellular particulate materials in the permissive cells, which fluoresce under ultraviolet light illumination when exposed to an appropriate dye.

3. The method of claim 1, in which the culturing is performed by:
(a) inoculating a permissive cell line with a sample; and (b) detecting the formation of extracellular particulate materials in the permissive cells, which fluoresce under ultraviolet light illumination when exposed to an appropriate dye

4. The method of claim 1, in which the permissive cells being used to detect the presence of a stealth virus are maintained in culture medium, from which the level of KELEA (Kinetic Energy Limiting Electrostatic Attraction) activity has been reduced by exposing the culture medium to sterile paper for a sufficient time prior to the medium being used to culture stealth viruses to have reduced the level of KELEA activity as assessed by the kinetic movement of lidocaine particles sprinkled onto the surface of the medium.

5. The method of claim 1, in which the permissive cells being used to detect the presence of a stealth virus are maintained in culture medium, which is replaced with fresh medium at more frequent intervals than required to maintain a sufficient supply of nutrients, but rather to reduce the levels of accumulating extracellular materials, which contribute KELEA activity to the medium; a suitable frequency of replacing the medium being 24 to 48 hours.

6. The method of claim 1, in which the intent on the culturing of the stealth virus in the permissive cells is to obtain cells and/or culture medium from a positive culture to for additional biochemical, immunological, and/or molecular-based assays to further characterize the components, including the genetic sequences, of the particular stealth virus being propagated in the permissive cells.

7. The method of claim 1, in which the permissive cells being used to detect the presence of a stealth virus, are maintained in serum-free culture medium.

8. The method of claim 1, in which the appropriate dye for use in staining the cultured permissive cells is neutral red dye at a concentration between 0.001 to 0.05%.

9. The method of claim 1, in which the appropriate dye for use in staining the cultured permissive cells is acridine orange dye at a concentration between 0.001 to 0.05%.

10. The method of claim 1, in which the appropriate dye for use in staining the cultured permissive cells is a dye other than neutral red dye or acridine orange dye and includes dyes, which are also easily visible under regular microscopy.

11. The method of claim 1 in which the sample is blood or cerebrospinal fluid collected from a patient with an illness that is causing neurological and/or psychiatric symptoms.

12. The method of claim 1 in which the sample is blood-derived mononuclear cells or is cerebrospinal fluid collected from a patient with the chronic fatigue syndrome.

13. The method of claim 1 in which the sample is blood-derived mononuclear cells or is cerebrospinal fluid collected from a child with autism or with other forms of behavioral and/or emotional disorders.

14. The method of claim 1 in which the sample is blood-derived materials, other than mononuclear cells, and include serum, plasma, polymorphonuclear cells, erythrocytes, and platelets

15. The method of claim 1 in which the sample is obtained from blood or blood products intended for transfusions into another individual.

16. The method of claim 1 in which the sample is obtained from tissue, including a tumor biopsy.

17. The method of claim 1 in which the sample is obtained from tissue, from an animal.

18. The method of claim 1 in which the permissive cells are of the same species as the source of the sample in which the presence of a stealth virus is being tested.

19. The method of claim 1 in which the permissive cells are of a different species from the source of the sample in which the presence of a stealth virus is being tested.

20. The method of claim 1 in which the sample is blood or cerebrospinal fluid collected from a patient with an autoimmune illness.
